(19) United States
(12) Patent Application Publication
(10) Pub. No.: US 2017/0157313 A1
(43) Pub. Date: Jun. 8, 2017

(54) ANYTIME MODIFY DOUCHE

(71) Applicants: Raymond Valrie, Los Angeles, CA (US); Leah Valrie, Los Angeles, CA (US)

(72) Inventors: Raymond Valrie, Los Angeles, CA (US); Leah Valrie, Los Angeles, CA (US)

(21) Appl. No.: 14/960,290

(22) Filed: Dec. 4, 2015

(51) Int. Cl. A61M 3/02

(52) U.S. Cl. CPC A61M 3/0262; A61M 2210/1475; A61M 2210/1483

(57) ABSTRACT

A feminine douche product packaged in a dual dispenser that simultaneously provides external and internal cleansing. The Anytime Modify Douche comprises a bifurcated dual dispenser nozzle, wherein the first nozzle is insertable into one's vagina to execute gentle internal cleansing and the second nozzle is directed to one's vulva to cleanse external genitalia. When the container is squeezed, douche product is dispensed from both nozzles simultaneously.

### CLAIM OF PRIORITY

This patent application claims priority under 35 USC 119 (e) (1) from U.S. Provisional Patent Application Ser. No. 62/087,401 filed Dec. 4, 2014, of common inventorship herewith entitled, “Anytime Modify Douche,” which is incorporated herein by reference as though the same were set forth in its entirety.

### FIELD OF THE INVENTION

The present invention pertains to the field of health and hygiene, and more specifically to the field of douche apparatuses.

### BACKGROUND OF THE INVENTION

The prior art has put forth several designs for douche apparatuses. Among these are:

U.S. Pat. No. 3,012,695 to Louis L. Lerner describes a container having a plurality of individual compartments secured together in a series. Each compartment has a flexible wall and is individually collapsible independently of the others by the application of external pressure. The compartment at one end of said container has an outlet opening directly to the outside of said container. Each of the remaining compartments has an outlet in its corresponding end, a conduit connected to each of the last said outlets extending to said one end through each of the intervening compartments. Each said conduit has a delivery end opening to the outside of said container adjacent the first said outlet. A conduit is connected to the outlet of the compartment at said one end of the container and communicates with a selected one of the remaining compartments. All of the remaining compartments also communicate with said selected compartment. A means for closing and opening each said outlet is provided to permit mixing of the contents of the several compartments within said selected compartment.

U.S. Pat. No. 858,996 to Edward John Lamport describes a nasal douche consisting of a container, a conducting tube connected with the bottom of the container, twin nozzles of nonflexible material attached to the bottom portion of the conducting tube. The said nozzles are upwardly curved at their free ends. Connected with the said conducting tube, the said nozzles have independent passages connecting them with said tube. A valve is common to both of said passages. Buttons at the tree ends of the nozzles are adapted for engagement with the nostrils of the user of the device.

U.S. Pat. No. 821,259 to Charles Pierre Theophile Roux describes an improved form of douche apparatus which enables persons using it to give themselves shower baths, jet douches, dorsal, pectoral, and all other kinds of douches. The douche apparatus chiefly comprises a flexible pipe a, made of rubber or the like and provided with a union piece which can be screwed or fixed in any other suitable manner to the tap of a water pipe. The other end of the pipe is suitably connected to a curved metal pipe. The pipe is provided with two handles of which one handle is adapted to operate a tap. The pipe is provided with two branches, each one having a tap. The short branch is secured to the straight portion intermediately of the same and extends laterally from said portion. The long branch or extension is curved away from the part at one end thereof in the direction in which the branch extends, but is curved backwardly at its free or discharge end, so as to oppose and be substantially aligned with said branch. The free or discharge ends of the branches are screw threaded to receive a jet nozzle or a rose.

None of these prior art references describe the present invention.

### SUMMARY OF THE INVENTION

It is an object of the present invention to provide a feminine douche packaged in a dual dispenser that simultaneously provides external and internal cleansing.

### BRIEF DESCRIPTION OF THE DRAWINGS

FIG. 1 is a prototypical illustrative side view of the present invention showing a container that holds a cleansing formulation and dual nozzles that comprise tiny holes for spraying the cleanser when the douche is manually squeezed.

FIG. 2 is a prototypical illustrative diagonal view of the present invention showing a container that holds a cleansing formulation, an elongated nozzle that inserts into a female's vagina, and a short angled nozzle that cleans the female's vulva area.

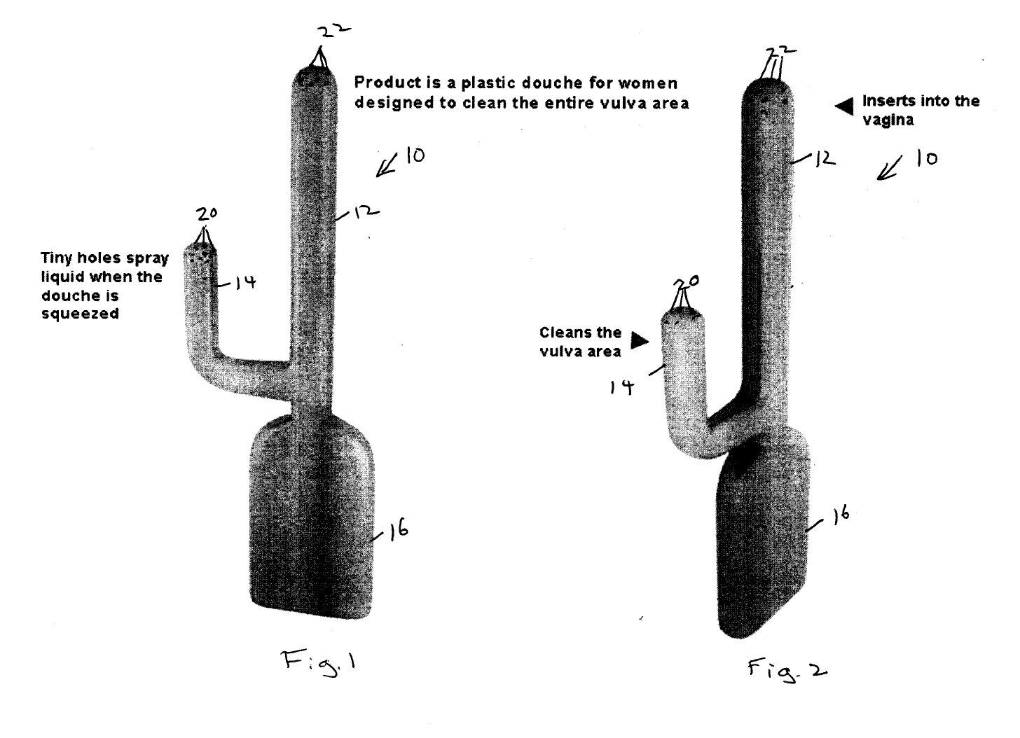

### DETAILED DESCRIPTION OF THE INVENTION

Proper hygiene is a key factor in maintaining a healthy and happy existence. Reducing germs and bacteria, hygiene rituals are important for making a person feel clean and fresh on a daily basis. Brushing one's teeth, taking a quick shower or enjoying a long, hot soak in the bathtub are all activities that cleanse the body and refresh the spirit. Although these habits provide an efficient means of cleansing the body and abating odors, trying to maintain that level of cleanliness throughout the day is challenging. In particular, maintaining a level of freshness is often difficult for females. A monthly period or occasional vaginal discharge can render one feeling uncomfortable and self conscious. Many females attempt to solve this problem by utilizing sanitary douches. These sanitary products are especially useful after a monthly period as they serve to bate odors and leave one feeling refreshed. These feminine douches are constructed for internal cleansing only.

The present invention, hereinafter referred to as the Anytime Modify Douche, is a feminine douche packaged in a dual dispenser that simultaneously provides external and internal cleansing. A practical modification to the current configuration of douches, the Anytime Modify Douche facilitates optimal feminine hygiene. Please refer to the drawings. The Anytime Modify Douche 10 comprises a bifurcated dual dispenser nozzle, the first nozzle 12 is inserted into one's vagina to execute gentle internal cleansing. The second nozzle 14 is directed to one's vaginal walls or vulva to cleanse external genitalia.

Formulated to quickly wash away trace residue while thwarting the growth of germs and bacteria, the Anytime Modify Douche is manufactured from a variety of gentle ingredients formulated to provide a practical way in which to cleanse the bodies following a monthly period, prior to or following sexual intimacy, or simply whenever one wants to cleanse themselves to feel more fresh. Unlike traditional prepackaged feminine douches comprising a single dispenser nozzle, the Anytime Modify Douche comprises a dual dispenser head.

The present invention's container is generally cylindrical in shape having an oval cross-section and comprised of a squeezable plastic bottle 16 measuring approximately six inches in height by two inches in diameter. Positioned at the top of the bottle are dispenser nozzles 12 and 14. Connected together to dispense product simultaneously, these dual dispenser nozzles 12, 14 include an elongated tubular nozzle 12 to be inserted into the vagina. A shorter nozzle 14 attached to the base of this nozzle extends outwards at an angle, or is positioned approximately two inches from the longer nozzle 12. The shorter nozzle 14 is sized appropriately to perform direct external cleansing of the vulva area of the female body. The end of each nozzle is rounded softly for optimal comfort and is comprises a plurality of small circular shaped egress openings 20, 22 through which cleansing liquid is dispensed when the squeezable container 16 is squeezed. A full size bottle of Anytime Modify Douche holds several ounces of fluid, ensuring complete and thorough cleansing of the body. The present invention is available in packages containing approximately four douches per container. An ingredient listing and instructions for use are clearly imprinted on the packaging.

Using the Anytime Modify Douche is very simple and straight forward. The Anytime Modify Douche is used whenever deemed appropriate such as following a menstrual period or simply if one wishes to enjoy a feeling of freshness. After removing the Anytime Modify Douche from the outer packaging, the user gives the bottle a gentle shake to further blend the ingredients. Squatting over or sitting on the toilet, the user holds the Anytime Modify Douche in one hand, directing the elongated straight nozzle into the vagina and positioning the present invention so the shorter and angled nozzle is directed at the external genitalia. Squeezing the bottle, the user applies enough pressure to administer a steady stream of the gentle cleansing solution, simultaneously cleansing their body internally and externally. After use, the empty Anytime Modify Douche is discarded in any waste receptacle.

The Anytime Modify Douche enables women to enjoy a more thorough and comfortable way in which to cleanse their body. Providing a dual cleaning tool, the Anytime Modify Douche provides an internal douche with the light, fresh cleansing of a bidet type spray, in one convenient package. Using the Anytime Modify Douche eliminates internal and external residue which causes discomfort, providing a hygienic means of maintaining freshness. The present invention's formulation easily washes away microscopic residue and effectively eliminates feminine odor, enabling a user to enjoy a sense of confidence and security throughout the day and into the evening. More than a mere body wash, the Anytime Modify Douche thwarts the spread of germs and bacteria to ensure optimal feminine health. The Anytime Modify Douche provides an efficient, convenient and hygienic means of cleansing the body both internally and externally.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A feminine douche product packaged in a dual dispenser that simultaneously provides external and internal cleansing, comprising:
a container of douche product generally cylindrical in shape having an oval cross-section and comprised of a squeezable plastic bottle, wherein the bottle comprises a pair of bifurcated dispenser nozzles comprising a first nozzle and a second nozzle wherein the nozzles are connected together to dispense douche product simultaneously from both nozzles.

2. The feminine douche product dispenser of claim 1 wherein the first nozzle is longer than the second nozzle, and the second nozzle is positioned approximately two inches from the first nozzle.

3. The feminine douche product dispenser of claim 1, wherein the terminal end of each nozzle is rounded softly for optimal comfort and comprises a plurality of small circular shaped egress openings through which douche product is dispensed when the squeezable plastic bottle is squeezed.

4. The feminine douche product dispenser of claim 3, wherein the second nozzle extends at an angle from the first nozzle.

5. The feminine douche product dispenser of claim 3, wherein the second nozzle is a smaller diameter than the first nozzle.

6. The feminine douche product dispenser of claim 1, wherein the squeezable container contains more than two ounces of douche product.

7. The feminine douche product dispenser of claim 3, wherein the squeezable container contains more than two ounces of douche product.

8. The feminine douche product dispenser of claim 1, available in packages containing approximately four douches per package.

9. The feminine douche product dispenser of claim 3, available in packages containing approximately four douches per package.

10. The feminine douche product dispenser of claim 1, wherein the container measures approximately six inches in height by approximately two inches in diameter.

11. The feminine douche product dispenser of claim 3, wherein the container measures approximately six inches in height by approximately two inches in diameter.
